(19) United States
(12) Patent Application Publication
(10) Pub. No.: US 2023/0165412 A1
(43) Pub. Date: Jun. 1, 2023

(54) FOOT CLEANING DEVICE

(71) Applicant: Grey Matters, LLC, Williston, VT (US)

(72) Inventor: Bruce Andrew Herberg, Williston, VT (US)

(21) Appl. No.: 17/997,289

(22) PCT Filed: Apr. 28, 2021
(86) PCT No.: PCT/US21/29611

§ 371 Date: Oct. 27, 2022

Related U.S. Application Data
(60) Provisional application No. 62/704,214, filed on Apr. 28, 2020.

(51) Int. Cl. A47K 7/02

(52) U.S. Cl. CPC A47K 7/026

(57) ABSTRACT

A person hygiene product includes a strap with loops on each end configured for grasping by the hand of a user and for being placed over the foot of the user. Between the loops and secured to the strap is a tubular segment of material suitable for scrubbing skin. In operation, one loop is placed over a foot while the other is grasped by a hand and the cloth segment is inserted between toes of the opposite foot for scrubbing. Afterward, the strap may be flipped, put onto the clean foot, and the procedure repeated for the other foot. In addition, both loops may be grasped in the hands of the user and the cloth segment may be passed back and forth over the bottom, sides, and top of the feet.

### FIELD OF THE INVENTION

The present invention generally relates to personal hygiene products for feet. In particular, the present invention is directed to a personal hygiene product that may be used as a foot cleaning device.

### BACKGROUND

There is not a simple tool to clean between the toes during regular bathing, whether in the shower or tub, without excessive and sometimes dangerous bending by the user, either at the back to reach down to the foot, or of the leg to lift it within reach. General products can be used with such bending required, and foot-specific devices are available but demand an allocation of space in often very constricted and potentially dangerous bathing areas. While toe gaps may be addressed during the drying of feet, there is a need for a device that facilitates the cleaning of feet, including between the toes, with water and an appropriate cleansing product.

### SUMMARY OF THE DISCLOSURE

A personal hygiene device has a first end and a second end, and includes a strap having a first strap end and a second strap end, a first loop at the first end of the device, wherein the first loop is formed by the securement of the first strap end to the strap and sized to be placed over a user's foot or grasped by the user's hand. A second loop at the second end of the device is formed by the securement of the second strap end to the strap and is sized to be placed over a user's foot or grasped by the user's hand. A tubular segment of soft cloth is further included, wherein the strap runs through the tubular segment of cloth such that the tubular segment of cloth covers the strap between the first loop and the second loop, and wherein the tubular segment of soft cloth is attached to the strap such that the tubular segment of cloth remains stationary with respect to the strap when in use for cleaning feet.

Additionally or alternatively, a first stitching secures the first strap end to the strap and a first end of the tubular segment of cloth to the strap and wherein a second stitching secures the second strap end to the strap and a second end of the tubular segment of cloth to the strap.

Additionally or alternatively, a third stitching secures the tubular segment of cloth to the strap and runs along a length of the tubular segment of cloth, forming a substantially flattened tube shape having a top surface, a bottom surface, a first folded edge, and a second folded edge opposite the first folded edge.

Additionally or alternatively, the tubular segment of cloth encompasses a portion of the first loop and a portion of the second loop.

In another aspect of the invention, a method of cleaning a foot of a user includes placing a first loop on one end of a strap over a first foot of the user, grasping a second loop on an opposite end of the strap in a hand of the user, pulling the strap sufficiently taut, inserting a portion of a tubular segment of cloth between toes of a second foot of the user, wherein the strap runs through the tubular segment of cloth such that the tubular segment of cloth covers the strap between the first loop and the second loop, and moving the second foot repeatedly up and down along the cloth, wherein the tubular segment of soft cloth is attached to the strap such that the tubular segment of cloth remains stationary with respect to the strap during the moving.

In another aspect of the invention, a method of cleaning a foot of a user includes grasping a first loop on one end of a strap in a first hand of the user, grasping a second loop on an opposite end of the strap in a second hand of the user, placing a portion of a tubular segment of cloth around a bottom of a foot of the user, wherein the strap runs through the tubular segment of cloth such that the tubular segment of cloth covers the strap between the first loop and the second loop, and moving the first loop and the second loop up and down, wherein the tubular segment of soft cloth is attached to the strap such that the tubular segment of cloth remains stationary with respect to the strap during the moving.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a perspective view of a personal hygiene device in accordance with an embodiment of the present invention;

FIG. 2 is a side view of the device shown in FIG. 1;

FIG. 3 is a cross sectional view through a portion of the device of FIG. 2;

FIG. 4 is a perspective view of a personal hygiene device in use to clean a foot of a user in which one handle portion is being grasped by the user and the opposite handle is positioned over a foot of the user while the opposite foot is scrubbed with the cloth portion of the device; and

FIG. 5 is a perspective view of personal hygiene device in use to clean a foot of a user in which both handles are grasped by the user and the bottom of the user's foot is scrubbed with the cloth portion of the device.

### DESCRIPTION OF THE DISCLOSURE

The invention is a strap with a soft cloth segment attached for cleaning the feet of a user, especially between the toes. The strap includes a loop on each end and has a length that allows a user to clean their feet while seated in a bathing area. Preferably, the loops are 7-12 inches from the end of the strap to the end of the loop while the strap have a length of 15-21 inches between the loops.

In a preferred embodiment, the strap may be a one-inch-wide flat piece of fabric, which may be polyester or other suitable material such as hemp. The strap includes a single thickness portion between two loops, which may be formed by folding the strap material over and sewing on both ends to create the two loops (which may be approximately nine-inches) and may have an overall length of about three feet, although other lengths will be appropriate. A snug-fitting sleeve or tube-shape of soft, one-sided, heavy-nap terry cloth, such as cotton or other suitable material that can be used to scrub or wash skin, encompasses the strap between the loops. In a preferred embodiment, the cloth sleeve is slightly longer than the single thickness portion of the strap, such as between 16 and 22 inches in length as required for the 15-21-inch length of the single thickness portion of the strap. The sleeve is set between the loops and sewn onto the strap across the width of each end to affix the cloth sleeve to the strap and to reinforce or create the loops described above. In addition, the cloth sleeve may also be sewn or otherwise attached at least once to the strap down its length to prevent the cloth sleeve from stretching and bunching during use.

In operation, the user wets the device. While holding the device out of the water, the user applies a personal cleansing product to both sides of the cloth sleeve. The user slips one of the loops over the front portion of one foot while holding the other loop in a hand. Then, with the strap pulled tight, the user positions the cloth segment between two toes of the loose foot and moves that foot up and down the soaped terry portion of the device. After a few strokes, the user repositions their knee to the other side of the strap and repeats the up/down strokes, ensuring both sides of the toe gap get scrubbed. In the preferred embodiments, the cloth sleeve is sized such that the user will only be utilizing half of the cloth sleeve while scrubbing one set of toes. The user then repositions the strap to clean between the next set of toes and repeats the process. Once all toe gaps are scrubbed, the loop from the stationary foot is moved to the hands and the loop previously held by the user's hands is put on the other foot and the procedure repeated for the unwashed foot. In this way, an unused section of the cloth sleeve is made available for the second foot.

The device can also be held by both hands (one hand grasping a respective one of the loops) and positioned under the foot. The device can be drawn back and forth width-wise under the foot to wash the bottom of the foot. It can also be wrapped around the foot and ankle in a z-like pattern to wash the sides and top of the foot.

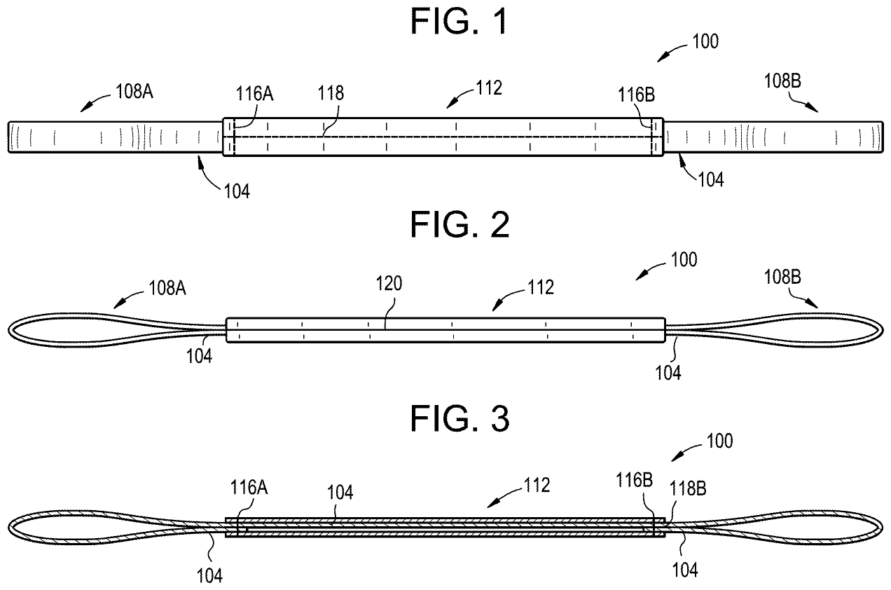

Turning to the figures, in FIGS. 1 and 2 a cleaning device 100 is shown that includes a strap 104 with a first handle 108A on one end and a second handle 108B on the opposite end. Between handles 108 is a tubular cloth segment 112 that surrounds and is secured to strap 104 such that cloth segment 112 remains stationary with respect to strap 104 during the foot cleaning procedure. In an embodiment, a lengthwise stitching portion 118 runs along the length of cloth segment 112 and secures cloth segment 112 to strap 104 between loops 108A-B. Preferably, stitching 118 engages with a center of a wide surface of strap 104, which forms two fabric folds 120 (e.g., 120A as can be seen in FIG. 2) on cloth segment 112 that are aligned with the narrow edges of strap 104 along the portion of device 100 where strap 104 and cloth segment 112 overlap.

Crosswise stitching 116 (e.g., 116A, 116B) runs across the width of both cloth segment 112 and strap 104 in order to both further secure cloth segment 112 to strap 104 and form loops 108A-B in a preferred embodiment. As can be seen in FIG. 3, strap 104 runs through the inside of tubular cloth segment 112 and loops 108 are formed by securing respective ends of strap 104 to strap 104. In this way, device 100 may be formed with a single length of strap, a single tubular cloth segment, and three stitch lines, although it will be recognized that other ways of constructing devices of the present invention may be used. In a preferred embodiment, cloth segment 112 partially overlaps with each of loops 108.

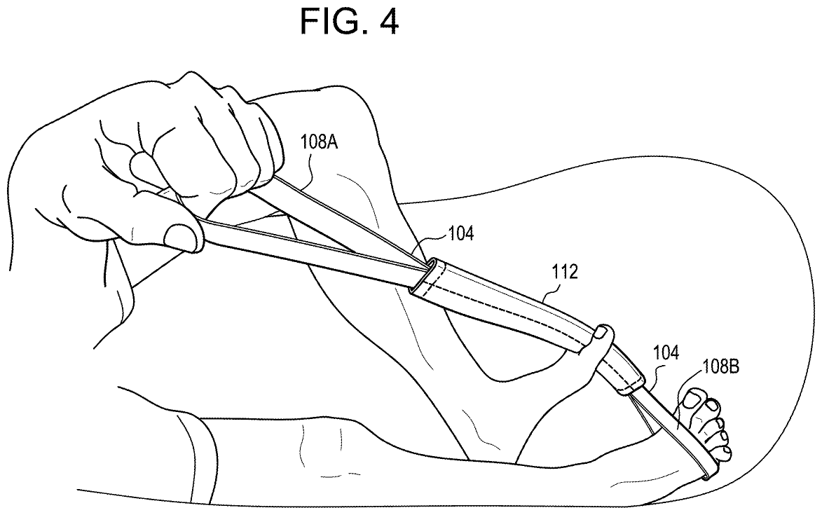

Cleaning device 100 can be used to more safely and effectively clean a user's feet. As described above and shown in FIG. 4, in an exemplary use a first loop is placed over a first foot of the user and the second loop is grasped by the user's hand. Then the cloth segment is placed between the toes of the opposite foot and a scrubbing motion is implemented. Once all between toes areas are cleaned, the loops are preferably switched so that the second loop is placed around the cleaned foot and the first loop is grasped by the user's hand. In this way, the portion of the cloth segment available for cleaning the toes of the second foot has not yet been used.

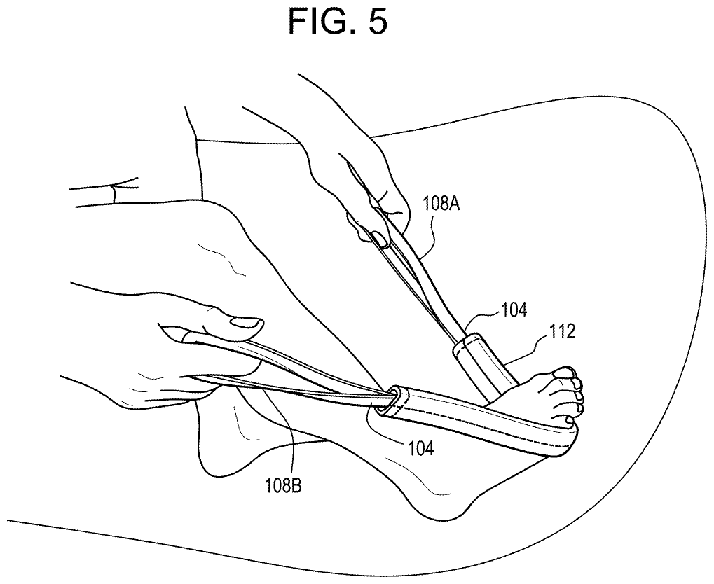

As shown in FIG. 5, in another exemplary use to clean the bottom and top of the feet, both loops may be grasped in the user's hands and the cloth segment pulled back and forth across one foot at a time.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A personal hygiene device having a first end and a second end, the device comprising:
a strap having a first strap end and a second strap end;
a first loop at the first end of the device, wherein the first loop is formed by the securement of the first strap end to the strap and sized to be placed over a user's foot or grasped by a hand of the user;
a second loop at the second end of the device, wherein the second loop is formed by securement of the second strap end to the strap and is sized to be placed over a user's foot or grasped by the hand of the user; and
a tubular segment of soft cloth, wherein the strap runs through the tubular segment of cloth such that the tubular segment of cloth covers the strap between the first loop and the second loop, and wherein the tubular segment of soft cloth is attached to the strap such that the tubular segment of cloth remains stationary with respect to the strap when in use for cleaning feet.

2. The personal hygiene device of claim 1, wherein the first loop is 9 inches in length.

3. The personal hygiene device of claim 1, wherein a first stitching secures the first strap end to the strap and a first end of the tubular segment of cloth to the strap and wherein a second stitching secures the second strap end to the strap and a second end of the tubular segment of cloth to the strap.

4. The personal hygiene device of claim 3, wherein a third stitching secures the tubular segment of cloth to the strap and runs along a length of the tubular segment of cloth, forming a substantially flattened tube shape having a top surface, a bottom surface, a first folded edge, and a second folded edge opposite the first folded edge.

5. The personal hygiene device of claim 4, wherein the tubular segment of cloth encompasses a portion of the first loop and a portion of the second loop.

6. A personal hygiene device having a first end and a second end, the device comprising:
a strap having a first strap end and a second strap end;
a first loop at the first end of the device sized to be placed over a user's foot or grasped by a hand of the user;
a second loop at the second end of the device sized to be placed over a user's foot or grasped by the hand of the user; and
a tubular segment of fabric, wherein the strap runs through the tubular segment of fabric such that the tubular segment of fabric encompasses the strap at least between the first loop and the second loop, and wherein the tubular segment of fabric is attached to the strap such that the tubular segment of fabric remains stationary with respect to the strap when the device is used to clean feet.

7. The personal hygiene device of claim 6, wherein the first loop is 9 inches in length.

8. The personal hygiene device of claim 6, wherein a first stitching secures a first strap end to the strap and a first end of the tubular segment of fabric to the strap and wherein a second stitching secures a second strap end to the strap and a second end of the tubular segment of fabric to the strap.

9. The personal hygiene device of claim 8, wherein a third stitching secures the tubular segment of fabric to the strap and runs along a length of the tubular segment of fabric such that the segment of fabric forms a substantially flattened tube shape having a top surface, a bottom surface, a first folded edge, and a second folded edge opposite the first folded edge.

10. The personal hygiene device of claim 6, wherein the tubular segment of fabric further encompasses a portion of the first loop and a portion of the second loop.

11. A method for cleaning a foot of a user comprising:
placing a first loop on one end of a strap over a first foot of the user;
grasping a second loop on an opposite end of the strap in a hand of the user;
pulling the strap sufficiently taut;
inserting a portion of a tubular segment of cloth between toes of a second foot of the user, wherein the strap runs through the tubular segment of cloth such that the tubular segment of cloth covers the strap between the first loop and the second loop; and
moving the second foot repeatedly up and down along the cloth, wherein the tubular segment of cloth is attached to the strap such that the tubular segment of cloth remains stationary with respect to the strap during the moving.

12. (canceled)
